(19) United States
(12) Patent Application Publication
(10) Pub. No.: US 2022/0330993 A1
(43) Pub. Date: Oct. 20, 2022

(54) IMPROVED CORTICO-CANCELLO-CORTICAL SCREW

(71) Applicant: Pravin SALUNKE, Chandigarh (IN)

(72) Inventor: Pravin SALUNKE, Chandigarh (IN)

(21) Appl. No.: 17/642,523

(22) PCT Filed: Sep. 26, 2019
(86) PCT No.: PCT/IN2020/050824

§ 371 Date: Mar. 11, 2022

(30) Foreign Application Priority Data
Sep. 27, 2019 (IN) 201911039180

(51) Int. Cl. A61B 17/86

(52) U.S. Cl. CPC A61B 17/863; A61B 17/8635

(57) ABSTRACT

An improved cortico-cancello-cortical screw (FIG. 8a-c) to hold properly in the cortical portion at the two ends of vertebral-body which has a threaded portion (L) provided with cortical thread for the cortical bone and cancellous thread for cancellous bone; a neck portion; a shoulder portion that connects the threaded portion to the screw head; a taper of approximately 2° along entire length of the screw to ensure smooth insertion and progress as the screw is fastened so that proximal portion of the screw would engage cortical bone the same way as the intermediate portion that would engage the cancellous bone. L is divided into L1 (proximal section) that engages the cortical bone of vertebra, L2 (intermediate section) that engages the cancellous bone and has flanges bent on itself and L3 (distal section) that engages distal cortical bone to provide increased surface area and better pull out strength.

### FIELD OF INVENTION

The invention generally relates to the field of Medical implants and orthopedic devices. More specifically, it relates to a cortico-cancello-cortical screw.

### BACKGROUND OF THE INVENTION

Bone screws are commonly used to attach plates, rods and other types of implants to one or more vertebrae, especially these have been used to achieve realignment and fusion in spinal surgeries.

### Definitions and Commonly Used Terms

Cancellous bone: The bone density along the surfaces of vertebrae is different from the central portion. The central portion has larger spaces and is relatively porous. This part is known as Cancellous bone, also known as spongy or trabecular bone. This is found at the ends of long bones, as well as in the pelvic bones, ribs, skull, and the vertebrae in the spinal column.

Cortical bone: Cortical bone is the dense outer surface of bone that forms a hard protective layer around the internal cavity. The bone is about 3 mm thick and also known as compact bone that makes up nearly 80% of skeletal mass and is imperative to body structure and weight bearing because of its high resistance to bending and torsion.

Bone Screw: A bone screw is a metal implant inserted into the bone. Screws are used to immobilize fractured bone segments to aid in the healing process, and as an adjunct to spine fusion surgery to help hold implants in place.

The cortical layer, though strong, is likely to fracture. Therefore a screw meant to hold the cortex has smaller flanges and shorter pitch, whereas, the porous cancellous bone requires screw with larger flanges and larger pitch to hold properly. In addition, the immature bone (pediatric) and osteoporotic bone (old age) have lesser density. Thus a screw with same thread geometry, pitch and core diameter cannot provide extraordinary pull out strengths, especially the immature bones or those with poor mineral density.

Pullout Strength: Pullout strength of screws is a parameter used to evaluate plate screw fixation strength. The pull out strength of a screw comes into play when the vertebrae are manipulated during surgery to achieve desirable alignment. Such alignment is required for:
Deformity correction—This occurs in young adults where the spine is grossly distorted in all 3 planes and is usually congenital. There is rotational, angular and translational malalignment in between the vertebral bodies. These are usually treated with inserting screws within the vertebral bodies and then manipulating them to achieve multiplanar correction in all planes. The maximum torques are borne by the vertebral bodies at ends of deformity. Such corrections require good pull out strengths/holding strength. This degree of manipulation is required for C1-2 congenital dislocation as well.
Traumatic listhesis (malalignment of vertebrae)—This occurs with trauma that disrupts the ligaments with fractures of weight bearing zones of vertebral bodies and connecting facets. Screws are required to align and unite the fractured fragments as well as to align the vertebral bodies. The manipulations and unions of fractures required good screw design that can hold the bone fragments during manipulation and later on till bony fusion occurs
Inflammatory lesion/neoplastic lesions of vertebral bodies (To hold adjacent bones in position without construct failure)—Malalignment can occur with inflammatory pathology. This diseased bone is removed and replaced with a metallic cage/mesh with bone grafts. It is important that the adjacent bones/vertebrae to the involved bone need to be fused for support. The screws inserted in these healthy vertebra play a pivotal role in holding the construct. With poor pull out strengths the construct would fail.
Degenerative listhesis or malalignment—There is dehydration of intervertebral tissues with aging, increasing the movements. To compensate this, the ends of vertebral bodies grow forming osteophytes. The instability pursues at times and is exaggerated by trauma or congenital variations. This leads to listhesis or dislocation between the vertebrae and compression of neural structures. Most of the patients with degenerative listhesis are in the older age group and have soft osteoporotic bones.

### Available Treatment and its Limitations

The vertebral bodies are manipulated, after performing release or wedge osteotomies and fused to each other with screws and rods/plates. Severe cases require insertion of screws and then manipulation for realignment. The screws and rods/plates need to hold the vertebra in aligned position till bony fusion occurs which ordinarily takes around 4-8 months. With some movements occurring at the adjacent spine, there is transmission of forces to the fixed zone and the stress is borne by the constructs.

Few cases are treated with mobility preserving devices such as artificial discs/joints/prosthesis. The Screws play a major role is holding the devices on the articulating surfaces till the time osteo-metallic integration (by definition, it is the direct structural and functional connection between a living bone and the surface of a load-bearing artificial implant made of metals) sets in. The screws are either designed to fit either cancellous or cortical bone. The problems occur at three steps:
1) While manipulating the vertebral bodies after insertion of screws or while tightening the rod into screw heads (tulips): During this process, there is a lot of angular (torque) and linear (pulling) forces acting between the bone-screw interfaces. It is obvious that the existing screws are likely to give away with excessive of these forces or with soft bones (as in children and older patients).
2) Construct failure before bony fusion sets in: Bony fusion usually takes around 4-8 months. There is lot of forces acting on the vertebral bodies as it cannot be immobilized unlike other bones of the body (limbs). This leads to constant forces on the screw-bone interface leading to construct failure.
3) With prosthesis: Screws play an important role in holding the implant in position while it provides adequate mobility. This produces a lot of pulling forces and torque on the screw-bone interface, especially at the extremes of movements. It is likely to get pulled out unless screws can hold till osseo-metallic integration occurs.

Screws with bicortical purchase (taking purchase within cortical bone on either side of vertebra) provide better pull out strength. However, these screws usually have a cortical thread through out and have poor hold in the larger cancellous portion. In addition, if the cortex is fractured, the pull out strength is significantly compromised. Recently screws with cortical thread towards its head with cancellous threads towards the tip have been made. They are called as the Cortico-cancellous screws. These may have a better holding strength, but are not bicortical and therefore not as effective in manipulation. The following screws with variable threads or tapering structures have been described in Prior art below.

### PRIOR ART

Patent Application No. US 20110276095A1 (Prior Art FIG. 1) discloses “Double Threaded Orthopedic screw”.

It relates to an orthopedic screw having a thread with two parts, a distal and a proximal part, each having a different thread configuration. The distal section has a thread with outer dimension and pitch suitable for entry into cancellous bone, while the proximal section has a composite thread comprising (i) a first thread of the same or slightly larger outer diameter as the cancellous thread in the distal section, having the same pitch thereof, and lying on the same helix, and (ii) another thread having a smaller outer diameter but the same pitch as the first thread, but disposed on a helix displaced from that of the first thread, such that it lies between the crests of the first thread. This screw enables optimum fixation strength in a bone or bones having a harder cortical outer section and a softer cancellous inner section. The screw may have an unthreaded central section.

The present invention shows a screw with tapered end with buttress thread for the cortical surfaces and the intermediate section for cancellous bone has threads with longer flanges with bent rims. The design is completely different from this published patent.
U.S. Pat. No. 5,871,486A (Prior art FIG. 2) discloses “Variable pitch bone screw”.

It relates to a bone screw having continuously varying pitch includes a tapered root portion having a relatively small diameter on a leading end of the screw and a larger trailing diameter. The pitch of the screw decreases between the leading and trailing ends thus causing the bone fragments in a fracture to be drawn together when the screw is installed across the fragments. Radially, the outer diameter of the threads increase between the leading and trailing ends thus causing each successive thread portion to cut into bone radially outwardly from the preceding thread portion thereby providing uncut bone in which the succeeding threads can gain purchase.

The present invention shows a screw with tapered end with buttress thread for the cortical surfaces and the intermediate section for cancellous bone. The screw disclosed in this prior art, showing the single threaded region and the thread is adopted to thread only in cancellous material. The design of the present invention is completely different from this published patent.
Patent Application No. US20160113693A1 (Prior art FIG. 3) discloses “Multi-thread bone screw and method”.

It relates to a bone screw comprising a threaded shank including a distal end portion and a proximal end portion, and defining a first threaded section extending from the distal end portion toward the proximal end portion and adapted for anchoring in cancellous bone. A second threaded section extends contiguously from the first threaded section toward the proximal end portion. The second threaded section has a finer thread pattern relative to the first threaded section. In one embodiment, the first threaded section includes a first helical threading defining a single lead thread pattern for anchoring in cancellous bone, and the second threaded section includes a second helical threading interleaved with the first threading to define a duel lead thread pattern for engagement in cortical bone. In a further embodiment, the bone screw includes a head portion extending from the threaded shank and configured for coupling to a spinal implant.

The present invention shows a screw with tapered end with buttress thread for the cortical surfaces and the intermediate section for cancellous bone has threads with longer flanges with bent rims. Whereas, the screw, disclosed in this prior art is showing only two threaded sections, instead of three. In addition, the geometry of threads is completely different from the published patent.
Patent Application No. US20140277188A1 (Prior art FIG. 4) discloses “Self-drilling, self-tapping bone screw and method of installing for bicortical purchase”.

It relates to a self-drilling, self-tapping bone screw in which the bone screw has a drill tip free of threads and having a length at least as great as about the thickness of a proximal cortical bone layer, with the drill tip having opposed lands and a helical flute between each of the lands with each of the lands having a cutting edge configured to cut bone as the drill tip is rotated into the bone with the flutes conveying the bone debris away from the drill tip, where a lead thread begins to self-tap internal threads in the proximal cortical bone layer after the drill tip has drilled through the proximal cortical bone layer so as to avoid stripping the threads formed in the bone layer. A method of installation is also disclosed.

The present invention shows a screw with tapered end with buttress thread for the cortical surfaces and the intermediate section for cancellous bone has threads with longer flanges with bent rims. Whereas, the screw, disclosed in this prior art is showing no tapering in cortical section and drill tip is free of threads. The design of present invention is completely different from this published patent.
U.S. Pat. No. 5,964,768 (Prior art FIG. 5) discloses “Tapered bone screw with continuously varying pitch”.

It relates to a bone screw having a continuously varying pitch includes a tapered root portion having a relatively small diameter on a leading end of the screw and a larger trailing diameter. The pitch of the screw decreases between the leading and trailing ends thus causing the bone fragments in a fracture to be drawn together when the screw is installed across the fragments. The radially outer diameter of the threads increases between the leading and trailing ends thus causing each successive thread portion to cut into bone radially outwardly from the preceding thread portion thereby providing uncut bone in which the succeeding threads can gain purchase.

The present invention shows a screw with tapered end with buttress thread for the cortical surfaces and the intermediate section for cancellous bone has threads with longer flanges with bent rims. Whereas, the screw, disclosed in this prior art is showing continuously decreasing pitch between the leading and trailing ends. The design of present invention is completely different from this published patent.
Patent Application No. CA2443880A1 (Prior art FIG. 6) discloses “Threaded washer”

It relates to an apparatus for the fixation of small bone fractures, comprising a washer with a central bore, the central bore having a diameter, and an external tapered thread for engaging an outer bone fragment, and a bone screw having a shaft with a thread with a mayor diameter less than the diameter of said central bore for engaging an inner bone fragment, and having a screw head larger than the central bore, wherein an upper portion near said screw head of the shaft is disposed within the central bore, and the screw head is disposed exterior the washer and engages a portion of the washer.

The present invention shows a screw with tapered end with buttress thread for the cortical surfaces and the intermediate section for cancellous bone has threads with longer flanges with bent rims. Whereas, the screw, disclosed in this prior art is showing a central bore with no thread. The design of present invention is completely different from this published patent.

Journals/Books
Stahel, P. F., Nicholas A. Alfonso, N. A, Henderson, C. and Baldini, T., 2017. Introducing the “Bone-Screw-Fastener” for improved screw fixation in orthopedic surgery: a revolutionary paradigm shift? Patient Safety in Surgery, 1146). DOI 10.1186/s13037-017-0121-5. (Prior art FIG. 7)

In this paper a newly designed Bone-Screw-Fastener was discussed that is based on an interlocking thread technology. This new fastener provides distributive forces from the threads onto the bone and therefore resists loads in multiple directions. The underlying concept is represented by a “female thread” bone cutting technology designed to maximize bone volume, preserve bone architecture, and create a circumferential interlocking interface between the implant and bone that protects the thread from stripping and from failing to multiaxial forces.

The present invention shows a screw with tapered end with buttress thread with differently varying pitch of threads. Whereas, the screw, disclosed in this prior art is showing thread with single type of pitch. The flanges have bent rim to counter forces in different direction. However the screw described does not have a taper. In addition the thread geometry is same throughout the length of screw. This is likely to fracture the cortical bone.

In contrast, the design described by us has a taper that allow gradual insertion of screw. Also, the threads are of buttress with larger core to flange diameter suitable for cortical bone. The bent rim flanges have a smaller core to outer diameter ratio (suitable for cancellous bone) but the core still is comparable to the tip section. Finally the section towards head again has flanges suitable for cortical bone and the core diameter is much bigger than the rest of screw. This would prevent any fracture of the cortical bone, allow an easy insertion and gain a good cancellous bone purchase. The design of present invention is completely different from this published paper.
Hsu, C. C., Chao, C. K., Wang, J. L., Hou, S. M., Tsai, Y. T., Lin, J., 2005. Increase of pullout strength of spinal pedicle screws with conical core: biomechanical tests and finite element analyses. Journal of Orthopaedic Research, 23, 788-794

This publication discloses that the screw loosening can threaten pedicle screw fixation of the spine. Conical screws can improve the bending strength, but studies of their pullout strength as compared with that of cylindrical screws have shown wide variation. In the present study, polyurethane foam with two different densities (0.32 and 0.16 gm/cm3) was used to compare the pullout strength and stripping torque among three kinds of pedicle screws with different degrees of core tapering. Three-dimensional finite element models were also developed to compare the structural performance of these screws and to predict their pullout strength. In the mechanical tests, pullout strength was consistently higher in the higher density foam and was closely related to screw insertion torque (r=0.87 and 0.81 for the high and low density foam, respectively) and stripping torque (r=0.92 and 0.78, respectively). Conical core screws with effective foam compaction had significantly higher pullout strength and insertion torque than cylindrical core screws (p<0.05). The results of finite element analyses were closely related to those of the mechanical tests in both situations with or without foam compaction. This study led to three conclusions: polyurethane foam bone yielded consistent experimental results; screws with a conical core could significantly increase pullout strength and insertion torque over cylindrical; and finite element models could reliably reflect the results of mechanical tests.

Here the authors have not disclosed any new design of screw but the experimental results. The authors suggest that screws with conical core have a better pull out strength. The flange design or thread geometry has not been evaluated.
Shea, T. M., Laun, J, Gonzalez-Blohm, S. A., Doulgeris, J. J., Lee III, W. E., Aghayev, K., Vrionis, F. D., 2014. Designs and Techniques That Improve the Pullout Strength of Pedicle Screws in Osteoporotic Vertebrae: Current Status. BioMed Research International. (Ref: http://dx.doi.org/10.1155/2014/748393)

This publication discloses different pedicle screw designs and instrumentation techniques that explored to enhance spinal device fixation in bone of compromised quality. These include alterations of screw thread design, optimization of pilot hole size for non-self-tapping screws, modification of the implant's trajectory, and bone cement augmentation. While the true benefits and limitations of any procedure may not be realized until they are observed in a clinical setting, axial pullout tests, and due in large part to their reproducibility and ease of execution, are commonly used to estimate the device's effectiveness by quantifying the change in force required to remove the screw from the body. The objective of this investigation is to provide an overview of the different pedicle screw designs and the associated surgical techniques either currently utilized or proposed to improve pullout strength in osteoporotic patients. Mechanical comparisons as well as potential advantages and disadvantages of each consideration are provided herein.

Here the authors have not disclosed any novel design of screw but discussed about different pedicle screw designs and instrumentation techniques to achieve better hold in the osteoporotic bone.
Ramaswamy, R., Evans, S., Kosashvili, Y., 2010. Holding power of variable pitch screws in osteoporotic, osteopenic and normal bone: Are all screws created equal? Injury, 41, 179-183.

In this publication, biomechanical properties of four different commercially available small fragment cannulated screws (Twin fix (Stryker, Freiburg, Germany), Herbert, (Zimmer, Warsaw, USA), Omnitech (Unimedical. Torino, Italy), Barouk (Depuy, Warsaw, USA)), with variable pitch, used for fracture fixation were compared. Polyurethane foam blocks of three different densities with mechanical properties similar to osteoporotic, osteopenic and normal bones were used to conduct the tests. Each screw was tested for pushout and pullout holding power after a primary insertion and for pullout after a repeated insertion into the respective foam blocks. The mean pullout and pushout strengths of all screws correlated to the foam density, and were significantly (p<0.001 and <0.001, respectively) better in foam with higher density. The mean pullout strength of each screw was consistently lower after reinsertion into the osteoporotic, osteopenic and normal bone densities by 4-30%, when compared to the index insertion. Yet, this difference was not found to be statistically significant (p=0.23). The Barouk screw performed significantly (p<0.0001) better than the other screws in all three different densities of foam for both for pushout and pullout after index insertion as well as for pullout tests after reinsertion. The holding power of screws is directly correlated to bone density, thread design and number of threads engaging the bone. Reinsertion through the same hole could reduce the ultimate pullout strength. The surgeon should consider the advantages and disadvantages of each implant, depending on the clinical situation and choose accordingly.

Here the authors have not disclosed any novel design of screw but the utility of different screw structures.

From the above, it is clear that there is no disclosure in the prior art regarding the tapered cortico-cancello-cortical screw with unique variable thread geometry.

### SUMMARY OF THE INVENTION

In the present invention, a newly designed screw has been developed that is tapered with buttress thread for the cortical surfaces and has flanges and pitch to hold appropriately in the cortical portion at the two ends. The intermediate portion for cancellous bone has threads with longer flanges with bent rims. The angulation of the threads on itself not only provides a larger surface area but also counters the torque and forces in various angles. This significantly would increase the holding strength and larger surface area is good for bones with relatively poor quality. This would help in greater manipulation without the fear of pull out to achieve good alignment. With this, the construct failure rates would decrease as well. The screw would be of benefit in bones with relatively poor density.

### BRIEF DESCRIPTION OF THE DRAWINGS

FIG. 1-FIG. 7 are prior art figures of existing Bone screws

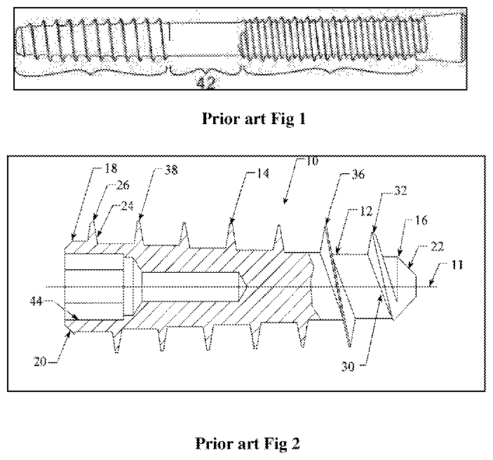

FIG. 8a—The section of vertebral body showing the cancellous center with outer cortical shell. In addition the entire screw as viewed from sides and top with inserts to further show each section of the screw

FIG. 8b—The detailed drawing of the screw showing thread, neck and shoulder

FIG. 8c—The detailed drawing of the screw showing the gentle tapering angle (2⁰) with dashed line

FIG. 9a—The proximal part of the threaded portion of the screw

FIG. 9b—The intermediate section of the threaded portion of the screw

FIG. 9c—The detailed flange anatomy of the L2 section of the screw

FIG. 9d—The distal portion of the screw

FIG. 9e—The flange anatomy of the L2 section of the screw in reverse direction

FIG. 10—The view of the screw-head as seen from top

### OBJECT OF THE PRESENT INVENTION

It is an object of the present invention to disclose a screw to properly hold the cortical portion at the two ends of injured bone.

Yet another object is to disclose a screw which is useful for bones with relatively poor quality and density.

One more object is to disclose a screw which can help in greater manipulation without the fear of pull out to achieve good alignment of the bone, so that the construct failure rates would drop.

### DETAILED DESCRIPTION OF THE INVENTION

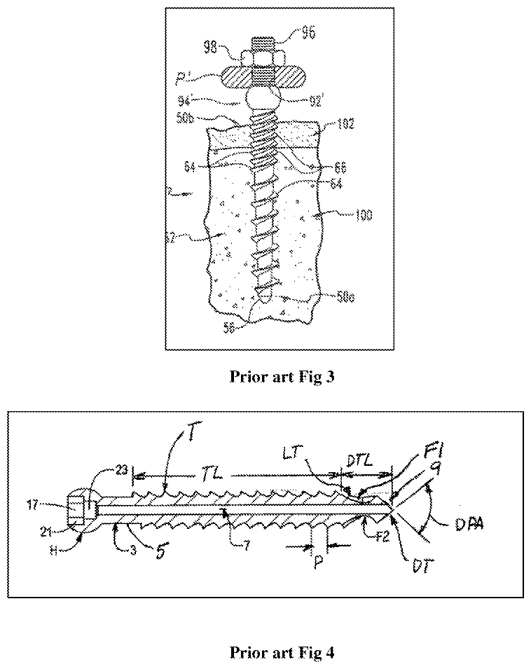

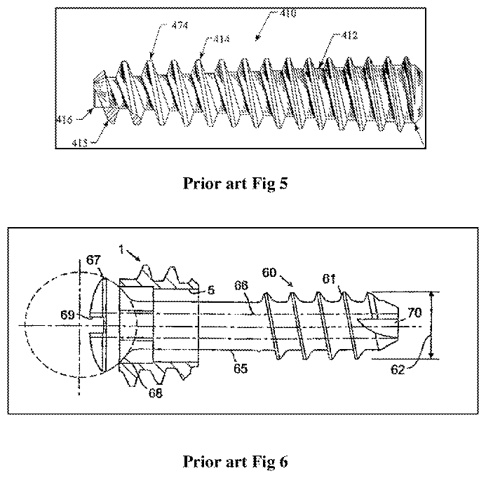

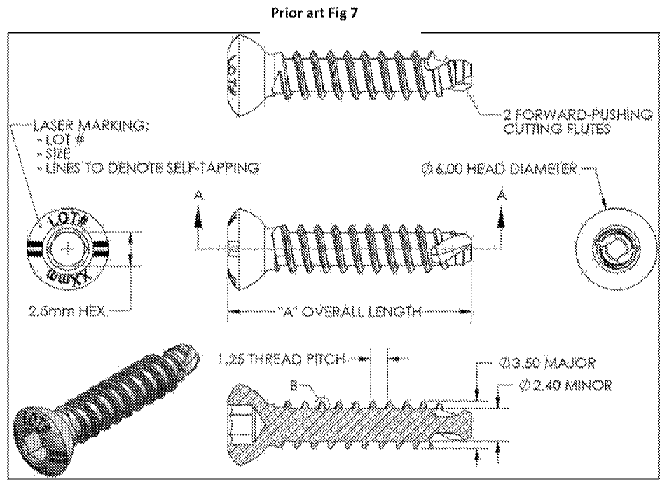

The present invention discloses a screw that has threads suitable for each particular part of the vertebral body; i.e., cortical thread for the cortical bone and cancellous thread for cancellous bone. FIG. 8a represents the vertebral body that has a central cancellous bone and has a peripheral cortical bone of approximately 3-5 mm thickness. The cortical bone is also seen along the pedicle and isthmus, the part that connects the vertebral body to the lamina. The picture of entire screw has been provided with various views. Referring to FIG. 8b, the screw is made of threaded portion marked by L the neck and shoulder that connects the threaded portion to the head. The threaded portion, L is further divided into 3 different segment. L 1 represent the distal most portion of the screw that engages the distal cortex of vertebral body whereas L 3 engages the proximal cortex of vertebral body. The intermediate section of the threaded portion, L 2 engages the cancellous bone. The length of L 1 is nearly constant irrespective of the vertebral level (cervical, thoracic or lumbar). The L1 is approximately 3-5 mm, the length of L3 depends on the vertebral level. For atlas or C1 lateral mass, the L 3 will be 3-4 mm. The same may be good for the subfacetal or pedicle screw. For pedicle screws below that level, L3 will be approximately equal to L2. The reason for this is subaxial spine, thoracic and lumbar spine have longer pedicles and proximal portion of the screw would engage cortical bone almost the same as the intermediate portion that would engage the cancellous bone. In general the screw has a gentle taper of approximately 2° along its entire length. This is depicted in the FIG. 8c with dashed lines. Prior drilling of the cortical portions of vertebrae is essential to avoid fracturing the cortex while fastening the screw. The taper along the entire length assures smooth insertion and progress as the screw is fastened. The outer diameter would be described for each portion (L 1-3) separately (vide infra). The pitch, P is separate for L 1, L 2 and L 3 and is described with each section. The flange (f) geometry is unique geometry for each section of the screw and has been described separately.

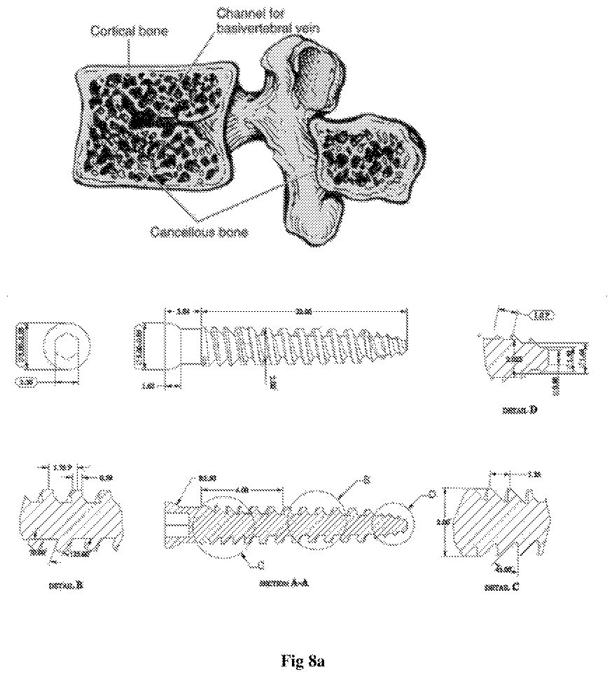

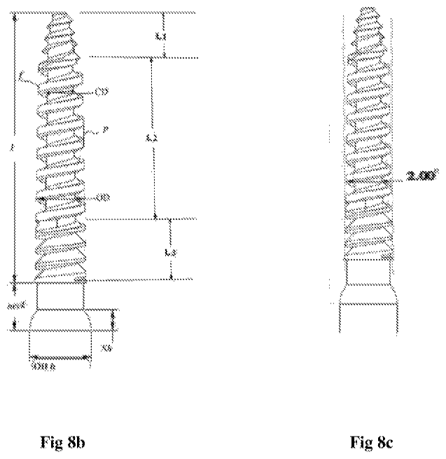

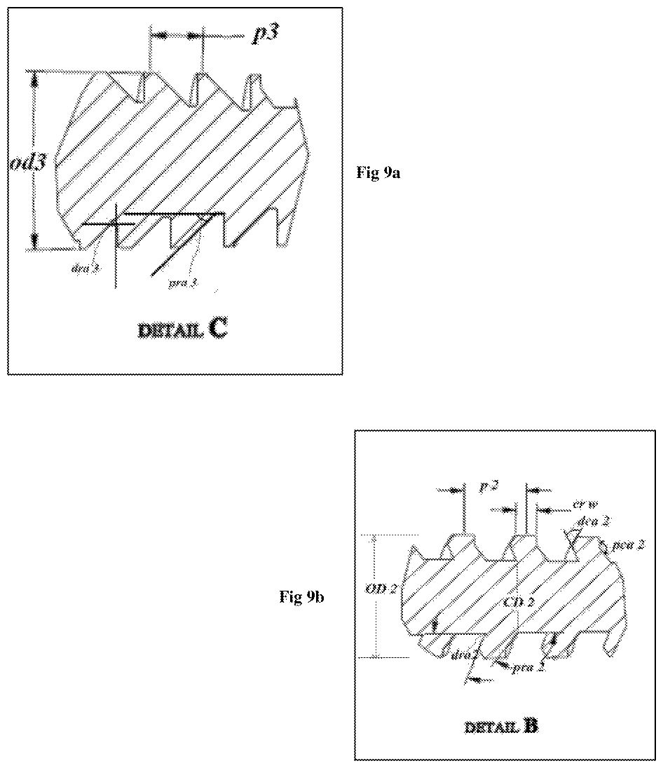

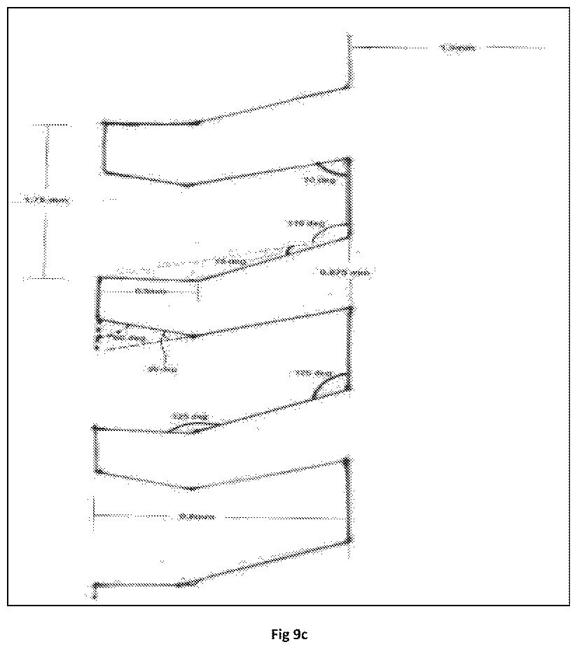

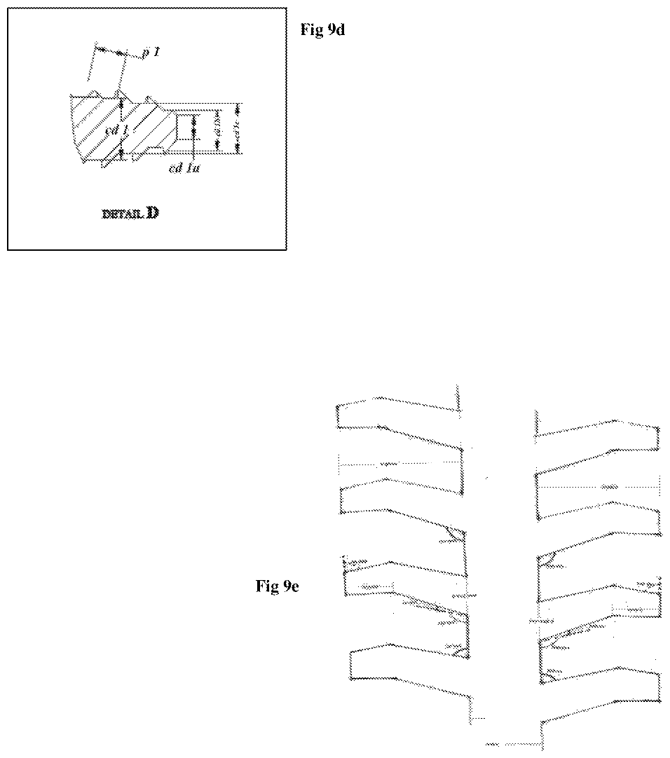

FIG. 9a represents the proximal part of the threaded portion that would engage the cortical bone of vertebra. The pitch (p3) or the distance between 2 adjacent flanges is 1.2-1.3 mm for smaller/thinner screw used in cervical spine and 1.4-1.6 mm for larger/thicker screw. The root is broader than the crest of the flange and is a typical buttress thread. The root forms an angle of approximately 45° with core proximally (pra3) and distal is 90° to the core (dra3). The Outer diameter (OD) of this portion depends on the vertebral segment where it would be used. For e.g. it would be 3.5 mm for upper cervical spine and 6.5 for the lumbar spine. Similarly the core diameter (CD) of this portion is 2.4 mm for cervical spine and approximately 4.5 mm for larger screws of lumbar spine. As described above the L 3 (length of this portion would depend on the vertebral body level where it would be used.

FIG. 9b represents the intermediate section of the threaded portion of the screw. The L 2 or the length would depend on the level of the vertebra. It would be around 14 mm for upper cervical spine and for subaxial spine and thoraco-lumbar spine it would approximately 14 mm to 30 mm. The outer diameter (OD) is 3.4 mm for upper cervical spine and 5-6.mm for larger lumbar spine. Core diameter (CD) is 2 mm for thinner screws for cervical spine and 2.8 to 3.5 mm for larger screws meant for lumbar spine. The pitch is 1.75 mm for thinner screws and approximately 2 mm for thicker screws. This portion would engage the softer cancellous bone and the thread geometry is the more complex. The flange anatomy is better described in FIG. 9c. The flange bend on itself at ⅔ length from the root. The proximal root angle (pra2) is 120-130° to core and the distal root angle (dra 2) is 65-75° to core. The proximal crest angle or pca (angle between proximal surface of flange edge and that towards the root) is 120-130° and the distal crest angle or dca (angle between under surface of flange edge and that toward the root) is 45-55°. The angle between the crest surface and flange undersurface is around 55-65°. The crest is approximately 0.5-0.8 mm thick

FIG. 9d represents the distal portion of the screw. The pitch (p1) is 0.85-0.95 mm for the smaller/thinner screws and 1.20-1.30 mm for larger/thicker screw. The length (L1) of this portion would be approximately 3 mm to 5 mm for cervical and thoraco-lumbar vertebra respectively. The Core diameter (CD) is rapidly tapering in this section from approximately 2-4 mm mm at proximally to 0.8 mm at the tip. The outer diameter (OD) is rapidly tapering as well from 3.4-5 mm proximally to 1.5 mm at the tip. The thread geometry is typical buttress one with proximal root angle of 40-50° (pra1) and distal root angle of 90° to the core (dra1). Adding cutting flutes to the tip would make the screw self-tapping.

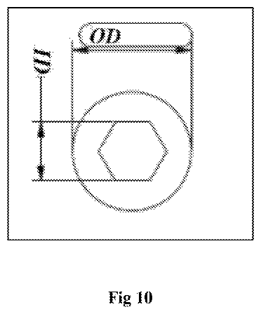

FIG. 10 represents the view of the screw-head as seen from top. The outer diameter would be 5.0 to 5.5 mm. The inner portion is a hexagonal notch that would engage in the screw driver.

The inventor being a neurosurgeon himself, has designed and developed a screw (FIG. 8a-c) that is tapered and has flanges (f, FIG. 8c, FIG. 9c) and pitch (p3, FIG. 9a) to hold appropriately in the cortical portion at the two ends. The proximal portion of the screw would engage cortical bone the same way as the intermediate portion that would engage the cancellous bone. The flanges in the intermediate portion have a larger surface area and are shaped in a manner to counter toggle in multiple axes. The screw is a tapered one with buttress thread for the cortical surfaces. The intermediate portion for cancellous bone has threads with longer flanges with bent rims. The angulation of the threads on itself not only provides a larger surface area but also counters the torque and forces in various angles. This significantly would increase the holding strength and larger surface area good for bone with poorer quality. This would help in greater manipulation without the fear of pull out to achieve good alignment.

The construct failure rates would drop too. The screw would be of benefit in bones with poorer density as well.

### NOVELTY, INVENTIVE STEP AND INDUSTRIAL APPLICATION

Novelty—In this present invention, a newly designed screw that is tapered and has flanges and pitch to hold properly in the cortical portion at the two ends of injured bone has been developed. This unique design would help in greater manipulation without the fear of pull out to achieve good alignment, so that the construct failure rates would drop. This unique idea has neither been disclosed nor anticipated by any of the prior art publications.

Inventive Step—The technical advancement of knowledge lies in the unique design of the screw wherein the intermediate portion for porous cancellous bone has threads with longer flanges with bent rims that provides a larger surface area, which is good for bones with relatively poor quality and density. The angulation of the threads counters the torque and forces in various angles to increase the holding strength. This would significantly help in greater manipulation without the fear of pull out to achieve good alignment. At the same time, the construct failure rates would drop. Therefore the present invention is technically more advanced than other available similar screws qualifying it for inventiveness.

Industrial application—The present invention can be easily manufactured on industrial scale and the production cost is not much as the raw material is cheap and readily available.

I claim:

1. An improved cortico-cancello-cortical screw (FIG. 8a-c) to hold properly in the cortical portion at the two ends of injured bone WHEREIN the screw has:
a threaded portion marked by L which is further provided with cortical thread for the cortical bone and cancellous thread for cancellous bone;
a neck portion;
a shoulder portion that connects the threaded portion to the screw head;
a taper of approximately 2° along entire length of the screw to ensure smooth insertion and progress as the screw is fastened; and
WHEREIN the proximal portion of the screw would engage cortical bone the same way as the intermediate portion that would engage the cancellous bone.

2. The improved cortico-cancello-cortical screw (FIG. 8a-c) as claimed in claim 1 WHEREIN the threaded portion L is divided into L1 (proximal section) the distal most portion of the screw that engages the cortical bone of vertebra, L2 (intermediate section) that engages the cancellous bone and L3 (distal section) that engages the distal cortical shell.

3. The improved cortico-cancello-cortical screw (FIG. 8a-c) as claimed in claim 1 WHEREIN the threaded portion L consists of flanges (f of FIG. 8c, FIG. 9c) of variable pitch (p3 of FIG. 9a) along its length such that the intermediate portion L 2 has longer flanges with bent rims that provides a larger surface area.

4. The improved cortico-cancello-cortical screw (FIG. 8a-c) as claimed in claim 1 WHEREIN the length of L 1 is 3-5 mm and the length of L 3 is 3-4 mm, L2 is 16-20 mm.

5. The improved cortico-cancello-cortical screw (FIG. 8a-c) as claimed in claim 1 WHEREIN the length of L 1 is 3-5 mm and the length of L3 is equal to L2 depending on the vertebral level, for pedicle screws at thoraco-lumbar region.

6. The improved cortico-cancello-cortical screw (FIG. 8a-c) as claimed in claim 1 WHEREIN proximal section L1 has following features:
the pitch (p3 of FIG. 9a) or the distance between 2 adjacent flanges is 1.20-1.30 mm for smaller/thinner screw used in cervical spine and 1.35-1.45 mm for larger/thicker screw used in lumbar spine;
the root is broader than the crest of the flange and is a buttress thread;
the root forms an angle of approximately 45° with core proximally (pra3) and distal is 90° to the core (dra3);
the Outer diameter (OD) of this portion is 3.0-3.5 mm for upper cervical spine and 6.0-6.5 for the lumbar spine;
the core diameter (CD) of this portion is 2.3-2.5 mm for cervical spine and 4.0-5.0 mm for larger screws of lumbar spine;

7. The improved cortico-cancello-cortical screw (FIG. 8a-c) as claimed in claim 1 WHEREIN intermediate section L 2 has following features:
the length of intermediate section L 2 (FIG. 9 c) of the threaded portion of the screw is 14 mm for upper cervical spine and 14 mm to 30 mm for subaxial spine and thoraco-lumbar spine;
the outer diameter (OD) is 3.0-3.6 mm for upper cervical spine and 5-6.5 mm for larger lumbar spine;
core diameter (CD) is 2 mm for thinner screws for cervical spine and 2.8 to 3.5 mm for larger screws meant for lumbar spine;
the pitch is 1.75 mm-2 mm for thinner/thicker screws;
the flange (FIG. 9 c) bends on itself at ⅔ length from the root;
the proximal root angle (pra2) is 120-130° to core and the distal root angle (dra 2) is 65-75° to core;
the proximal crest angle (pca—angle between proximal surface of flange edge and that towards the root) is 120-130′ and the distal crest angle (dca—angle between under surface of flange edge and that toward the root) is 45-55°;
the angle between the crest surface and flange undersurface is 60°-62°; and
the crest is 0.5-0.8 mm thick.

8. The improved cortico-cancello-cortical screw (FIG. 8a-c) as claimed in claim 1 WHEREIN distal section L3 (FIG. 9d) has following features:
the length of this portion would be approximately 3 mm to 5 mm for cervical and thoraco-lumbar vertebra respectively;
the pitch (p1) is 0.9 mm for the smaller/thinner screws and 1.25 mm for larger/thicker screw;
the core diameter (CD) is rapidly tapering in this section from approximately 2-4 mm at proximally to 0.8 mm at the tip;
the outer diameter (OD) is rapidly tapering as well from 3.4-5 mm proximally to 1.5 mm at the tip;
the thread geometry is buttress with proximal root angle of 45° (pra1) and distal root angle of 90° to the core (dra1);
adding cutting flutes to the tip would make the screw self-tapping;

9. The improved cortico-cancello-cortical screw (FIG. 8a-c) as claimed in claim 1 WHEREIN direction of the flanges of L2 is vertically reversible (FIG. 9c and FIG. 9e).

10. The improved cortico-cancello-cortical screw (FIG. 8a-c) as claimed in claim 1 WHEREIN the outer diameter of the screw head is at least 5.0 mm and more for the thoraco-lumbar spine.
